(19) United States
(12) Patent Application Publication
(10) Pub. No.: US 2024/0293043 A1
(43) Pub. Date: Sep. 5, 2024

(54) MONITORING PERSONS IN A ROOM

(71) Applicant: SenterCare Ltd., Misgav (IL)

(72) Inventors: Nir Goldenberg, Zoran (IL); Avshalom Shenhav, Haifa (IL); Arnon Hadas, Moshav Avigdor (IL); Yaakov Greenberg, Even Yehuda (IL); Shahar Harari, Tel Aviv (IL)

(73) Assignee: SenterCare Ltd., Misgav (IL)

(21) Appl. No.: 18/261,699

(22) PCT Filed: Dec. 13, 2021
(86) PCT No.: PCT/IB2021/061631

§ 371 Date: Jul. 17, 2023

Related U.S. Application Data
(60) Provisional application No. 63/138,576, filed on Jan. 18, 2021.

(51) Int. Cl. A61B 5/11; A61B 5/00; A61B 5/117; G08B 21/04; G16H 10/60; G16H 15/00

(52) U.S. Cl. CPC A61B 5/1126; A61B 5/1113; A61B 5/117; A61B 5/6802; A61B 5/6889; G08B 21/0423; G16H 10/60; G16H 15/00; A61B 2503/08; A61B 2562/0252

(57) ABSTRACT

A system for monitoring individuals includes sensors configured to collect data over a period of time. The data is associated with physical characteristics and behavioral characteristics of an individual in a room over the period of time. A processor is in communication with the sensors and coupled to an artificial intelligence processor device, which learns the physical characteristics and behavioral characteristics and defines therefrom normal physical characteristics and normal behavioral characteristics for the individual. The processor can compare physical characteristics and behavioral characteristics of the individual associated with other data, collected over another period of time from the sensors, with the normal physical behavioral characteristics and provide a report of the comparison.

### FIELD OF THE INVENTION

The present invention relates generally to monitoring systems, and particularly to a system or method for monitoring persons (e.g., elderly persons) in a room, which distinguishes between individuals and learns their behavior.

### BACKGROUND OF THE INVENTION

Many systems have been proposed and developed for tracking or monitoring human beings, such as elderly people in homes, hospitals and other buildings and settings.

Such systems may include sensors worn by the individual, such as motion sensors (e.g., accelerometers), and biological parameter sensors (e.g., pulse, blood pressure, sweat, etc.). Radars of different energy types and wavelengths have been used to monitor the movement of the individuals, but if there is more than one person in the room, it is difficult to distinguish between them.

Sensors have been used to monitor and alert the occurrence of falls or other accidents in the bathroom, kitchen and other rooms. Sensors have been used to monitor sleep patterns, such as motion sensors and load cells installed in a bed, couch and the like.

Sensors have been used to monitor living patterns in and out of the home, such as normal and abnormal times spent in the bathroom, bedroom or living room.

However, there still remains a need for improved non-intrusive monitoring of different elderly persons in a room.

### SUMMARY

The present invention seeks to provide a system/method for monitoring persons (e.g., elderly persons) in a room, which distinguishes between individuals and learns their behavior, as is described more in detail hereinbelow. The invention is particularly applicable for home use, but can be used in any setting, such as a hospital or senior citizen domicile.

The system combines different sensors with AI (artificial intelligence) or machine learning to provide improved elderly care monitoring capabilities. The system can analyze large scale behavioral data collected at the homes of elderly people. The system analyzes the data, together with the person's medical records and historical patterns, and constantly provides alerts and recommendations to health care providers or care call centers, enabling them to provide better, smarter care.

The system can unambiguously differentiate between different individuals, such as differentiating between an elderly husband and his elderly wife and any other persons, such as aides, inhabitants, visitors, etc., that are currently in the room.

The system can take into account clinical records of the monitored individual, matching them with the particular individual and monitoring the clinical history of that individual to provide valuable information to healthcare personnel regarding changes in the behavior and well-being of the individual. The system thus provides comprehensive monitoring of the activities of daily living (ADL) of the individuals.

The system may use time-of-flight (ToF) sensors and/or load cells or other sensors for detection of any anomaly in the ADL of the individuals. The ToF sensors may be used to unambiguously differentiate between different individuals. The ToF sensors can be used to measure distances, shapes and volumes, such as anatomical features (e.g., head or any other limb size, proportions of limb size, etc.), movement features (e.g., the particular gait or other walking feature or gestures unique to the individual), or tracking the whereabouts of the individual and more.

The monitoring system of the invention can monitor activities of individuals and determine when the individual is in distress and communicate an alarm to appropriate personnel. The system may include sensors worn by the individual, such as motion sensors (e.g., accelerometers), and biological parameter sensors (e.g., pulse, blood pressure, sweat, etc.).

### BRIEF DESCRIPTION OF THE DRAWINGS

The present invention will be understood and appreciated more fully from the following detailed description taken in conjunction with the drawings in which:

FIG. 1 is a simplified block diagram illustration of a monitoring system and method, in accordance with a non-limiting embodiment of the present invention; and

FIG. 2 is a simplified illustration of different non-limiting components of the system/method.

### DETAILED DESCRIPTION

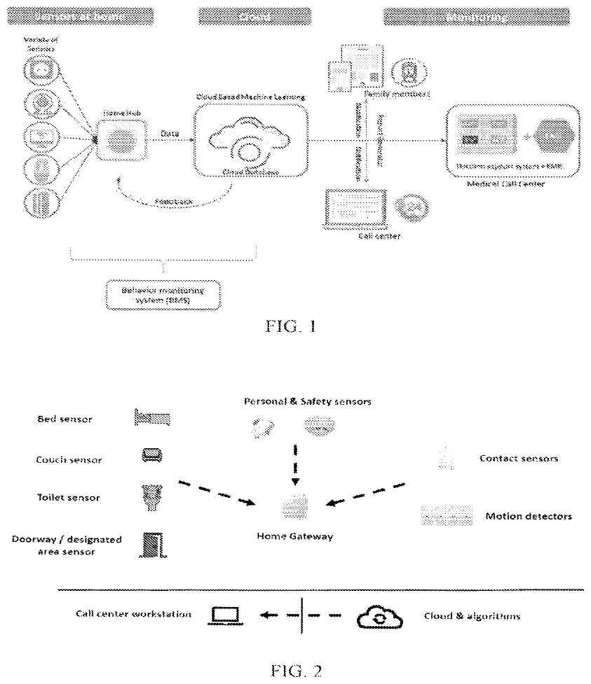

Reference is now made to FIG. 1, which illustrates a monitoring system and method, in accordance with a non-limiting embodiment of the present invention.

The system may include a variety of sensors situated in a room which are in communication with a processor, such as a home hub or cloud-based processor. The home hub or processor may be coupled to an artificial intelligence processor device, such as a cloud-based machine learning device or system.

Some of the sensors may include time-of-flight (ToF) sensors and/or load cells or other sensors for detection of any anomaly in the ADL of the individuals. The ToF sensors may be used to unambiguously differentiate between different individuals. The ToF sensors can be used to measure distances, shapes and volumes, such as anatomical features (e.g., head or any other limb size, proportions of limb size, etc.), movement features (e.g., the particular gait or other walking feature or gestures unique to the individual), or tracking the whereabouts of the individual and more.

The monitoring system of the invention can monitor activities of individuals and determine when the individual is in distress and communicate an alarm to appropriate personnel, such as family members, a call center or a medical call center which includes a decision support system and emergency medical responders. The system may include sensors worn by the individual, such as motion sensors (e.g., accelerometers), and biological parameter sensors (e.g., pulse, blood pressure, sweat, etc.).

As seen in FIG. 2, the sensors may include, without limitation, bed or couch sensors (e.g., load cells that sense the presence of the person sitting or lying on the bed or couch), toilet sensors (e.g., load cell that senses the presence of the person sitting on the toilet seat), doorway or designated area sensor (e.g., camera, electric eye, ToF sensor that detects the passage or presence of a person), contact sensors (e.g., capacitance sensors), motion detectors (e.g., ToF sensor or accelerometer), and personal and safety sensors (e.g., biological parameter sensors, smoke detectors, fire detectors, and others). The call center may have workstations in communication with cloud-based databases and processors.

The sensors may provide data about the individual being monitored, such as without limitation, time of entering or leaving home or room, total time in room, bed, couch, toilet, etc., anomalies such as abnormal duration or number of times in bed or toilet, etc.

In general, the sensors collect data associated with physical characteristics and behavioral characteristics of the individual in the room over a period of time. The data is transferred to the processor, which is coupled to the artificial intelligence processor device. The artificial intelligence processor device may be used to learn the physical characteristics and the behavioral characteristics and define therefrom normal physical characteristics and normal behavioral characteristics for the individual. Afterwards, the system collects other data over another period of time from the sensors and uses the processor to compare physical characteristics and behavioral characteristics of the individual associated with the other data with the normal physical characteristics and normal behavioral characteristics and provide a report of the comparison.

The report may include an alert or recommendation to a health care provider or a care call center.

The system can sense a presence of another individual in the room and use the processor to unambiguously differentiate between the individual and the other individual such that the normal physical characteristics and normal behavioral characteristics for the individual are not influenced by the other individual.

The processor may process the data and the other data by taking into account a medical record or stored historical pattern of the individual to generate valuable information to the health care provider. The report includes activities of daily living (ADL) of the individual.

What is claimed is:

1. A method for monitoring individuals comprising:
collecting data over a period of time from sensors situated in a room, said data being associated with physical characteristics and behavioral characteristics of an individual in said room over said period of time;
transferring said data to a processor coupled to an artificial intelligence processor device;
using said artificial intelligence processor device to learn said physical characteristics and said behavioral characteristics and define therefrom normal physical characteristics and normal behavioral characteristics for said individual; and
collecting other data over another period of time from said sensors and using said processor to compare physical characteristics and behavioral characteristics of said individual associated with said other data with said normal physical characteristics and normal behavioral characteristics and provide a report of said comparison.

2. The method according to claim 1, further comprising using at least one of said sensors to sense a presence of another individual in said room and using said processor to unambiguously differentiate between said individual and said other individual such that said normal physical characteristics and normal behavioral characteristics for said individual are not influenced by said other individual.

3. The method according to claim 1, wherein said processor processes said data and said other data by taking into account a medical record or stored historical pattern of said individual.

4. The method according to claim 1, wherein said report comprises an alert or recommendation to a health care provider or a care call center.

5. The method according to claim 1, wherein said report comprises activities of daily living (ADL) of said individual.

6. The method according to claim 1, wherein at least one of said sensors comprises a time-of-flight (ToF) sensor.

7. The method according to claim 6, comprising using said ToF sensor to measure anatomical features or movement features unique to said individual.

8. The method according to claim 1, wherein at least one of said sensors comprises a load cell.

9. The method according to claim 1, wherein at least one of said sensors comprises a biological parameter sensor worn by said individual.

10. The method according to claim 1, wherein at least one of said sensors comprises a motion sensor worn by said individual.

11. The method according to claim 1, wherein at least one of said sensors comprises a sensor mounted on an object in said room.

12. The method according to claim 1, wherein said artificial intelligence processor device comprises a cloud-based machine learning device.

13. A system for monitoring individuals comprising:
sensors configured to collect data over a period of time, said data being associated with physical characteristics and behavioral characteristics of an individual in a room over said period of time; and
a processor in communication with said sensors and coupled to an artificial intelligence processor device, wherein said artificial intelligence processor device is configured to learn said physical characteristics and said behavioral characteristics and define therefrom normal physical characteristics and normal behavioral characteristics for said individual, and said processor is configured to compare physical characteristics and behavioral characteristics of said individual associated with other data, collected over another period of time from said sensors, with said normal physical characteristics and normal behavioral characteristics and provide a report of said comparison.

14. The system according to claim 13, wherein at least one of said sensors comprises a time-of-flight (ToF) sensor.

15. The system according to claim 13, wherein at least one of said sensors comprises a load cell.

16. The system according to claim 13, wherein at least one of said sensors comprises a biological parameter sensor worn by said individual.

17. The system according to claim 13, wherein at least one of said sensors comprises a motion sensor worn by said individual.

18. The system according to claim 13, wherein at least one of said sensors comprises a sensor mounted on an object in said room.

19. The system according to claim 13, wherein said artificial intelligence processor device comprises a cloud-based machine learning device.
